(19) United States
(12) Patent Application Publication
(10) Pub. No.: US 2019/0357885 A1
(43) Pub. Date: Nov. 28, 2019

(54) ULTRASONIC PROBE AND ULTRASONIC DIAGNOSIS DEVICE

(71) Applicant: Konica Minolta, Inc., Tokyo (JP)

(72) Inventors: Hideo HONGO, Yokohama-shi (JP); Takehiko SUGINOUCHI, Tokyo (JP)

(21) Appl. No.: 16/404,105

(22) Filed: May 6, 2019

(30) Foreign Application Priority Data
May 24, 2018 (JP) 2018-099251

(51) Int. Cl. A61B 8/00

(52) U.S. Cl. CPC A61B 8/461; A61B 8/4444; A61B 8/565; A61B 8/4438; A61B 8/546

(57) ABSTRACT

There is disclosed an ultrasonic probe which transmits and receives an ultrasonic wave, including
a vibrator which transmits the ultrasonic wave in response to a transmission signal, and generates a reception signal of the reflected ultrasonic wave,
at least one abnormality detector which is provided in the ultrasonic probe, and detects an abnormality and indicates the abnormality in color, and
a state monitor which is provided in the ultrasonic probe, and monitors a state of the abnormality detector by the color, and outputs monitoring information.

### BACKGROUND

### Technological Field

The present invention relates to an ultrasonic probe and an ultrasonic diagnosis device.

### Description of the Related Art

Heretofore, an ultrasonic diagnosis device has been known in which an internal structure of a subject is irradiated with an ultrasonic wave by use of an ultrasonic probe, and the reflected ultrasonic wave is received and subjected to predetermined signal data processing, to generate an ultrasonic image of the internal structure of the subject. Such an ultrasonic diagnosis device is broadly for use in various use applications of inspection for medical purposes, a medical treatment, inspection of an interior structure of a building construction, and the like.

The ultrasonic probe is detachably attached to a main body of the ultrasonic diagnosis device, and inserted (connected) or extracted (disconnected) by an operator. For example, at this time, there is concern that failure or change of characteristic occurs, if an impact is applied to the ultrasonic probe due to fall, collision or the like. To solve this problem, there is known an ultrasonic probe including an irreversible impact detection element which detects the impact. When the impact is detected by the irreversible impact detection element, an operation of the ultrasonic probe is stopped, and this stop is notified to the ultrasonic diagnosis device (Japanese Patent No. 3315936). The irreversible impact detection element is operated only by using the impact, and the impact can be therefore detected also when the ultrasonic probe is unused or stored.

Furthermore, there is known an ultrasonic probe including a grip case, an impact detection member which indicates detection of an impact by change of a color or change of a shape, a holding member which holds the impact detection member in the grip case, and a window member provided in an outer wall of the grip case to confirm the change of the color or the change of the shape detected by the impact detection member from outside (Japanese Patent Laid-Open No. 2015-97626).

Additionally, in the ultrasonic probe described in Japanese Patent No. 3315936, the irreversible impact detection element includes a glass tube and colored ink enclosed in this glass tube. It is detected that the colored ink is diffused due to the impact, by detecting that the impact is applied to the impact detection member, when an amount of light output from a light emitting diode and transmitted through the irreversible impact detection element decreases and an amount of the light received by a phototransistor decreases. However, in this method, it is necessary for an ink diffusing portion to sufficiently transmit the light from the light emitting diode before the ink is diffused due to the impact. Furthermore, the diffusion of the colored ink cannot be detected until the impact that is not less than a sensor detection value is applied and a transmitting portion is obstructed by the diffusion of the ink after elapse of predetermined time. Additionally, a state of the irreversible impact detection element cannot be seen.

In the ultrasonic probe described in Japanese Patent Laid-Open No. 2015-97626, it is necessary to provide the window member for monitoring in the outer wall of the grip case, so that a structure of the grip case becomes complicated. Furthermore, for example, there is concern that the color of the window member for monitoring changes due to disinfection of the window member. Additionally, an operator who operates the probe visually judges and determines the color change of the window member or the like, and a large burden is imposed on the operator. Additionally, if the operator misses the impact detection, the ultrasonic probe having a possibility of failure due to the impact might be used in ultrasonic diagnosis, and an operator of ultrasonic diagnosis might make a wrong diagnosis to a patient who undergoes the ultrasonic diagnosis.

### SUMMARY

An object of the present invention is to easily and securely detect an abnormality such as an impact on an ultrasonic probe.

To achieve at least one of the abovementioned objects, according to an aspect of the present invention, an ultrasonic probe reflecting one aspect of the present invention comprises an ultrasonic probe which transmits and receives an ultrasonic wave, comprising

a vibrator which transmits the ultrasonic wave in response to a transmission signal, and generates a reception signal of the reflected ultrasonic wave,

at least one abnormality detector which is provided in the ultrasonic probe, and detects an abnormality and indicates the abnormality in a color, and

a state monitor which is provided in the ultrasonic probe, and monitors a state of the abnormality detector by the color, and outputs monitoring information.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is an overall view of a first ultrasonic diagnosis device of a first embodiment of the present invention;

FIG. 2 is a block diagram showing a functional constitution of the first ultrasonic diagnosis device;

FIG. 3A is a schematic view of an impact detection element before an impact is applied;

FIG. 3B is a schematic view of the impact detection element after the impact is applied;

FIG. 4 is a view showing a first sensor unit;

FIG. 5 is a diagram showing a configuration of a color sensor;

FIG. 6 is a flowchart showing first abnormality detection processing;

FIG. 7 is a block diagram showing a functional configuration of a second ultrasonic diagnosis device;

FIG. 8A is a view showing an ultrasonic probe having a coupling at a left rotation position;

FIG. 8B is a view showing the ultrasonic probe having the coupling at a right rotation position;

FIG. 9A is a view showing a pin at the left rotation position;

FIG. 9B is a view showing the pin at the right rotation position;

FIG. 10 is a block diagram showing a functional configuration of a third ultrasonic diagnosis device;

FIG. 11 is a block diagram showing a functional configuration of a fourth ultrasonic diagnosis device;

FIG. 12 is a block diagram showing a functional configuration of a fifth ultrasonic diagnosis device;

FIG. 13A is a view showing a second sensor unit;

FIG. 13B is a view showing a third sensor unit;

FIG. 14 is a flowchart showing second abnormality detection processing; and

FIG. 15 is a view showing an ultrasonic probe in a state where a frame is deformed.

### DETAILED DESCRIPTION OF EMBODIMENTS

Hereinafter, one or more embodiments of the present invention will be described with reference to the drawings. However, the scope of the invention is not limited to the disclosed embodiments.

First to fifth embodiments according to the present invention will be described in order in detail with reference to the accompanying drawings.

### First Embodiment

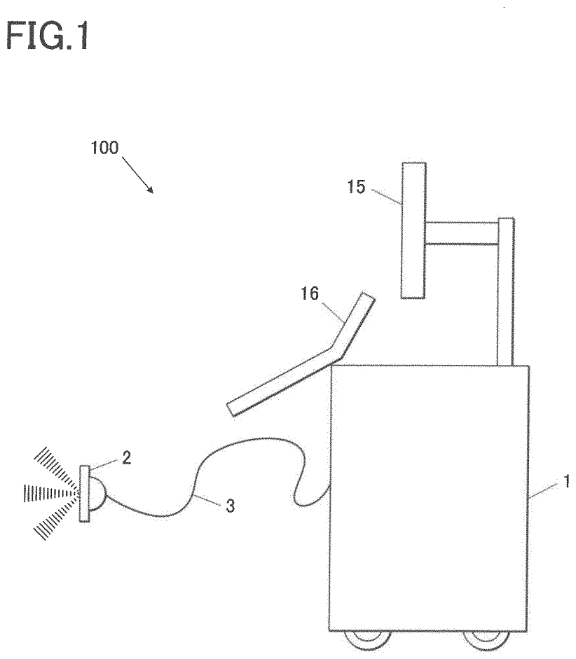

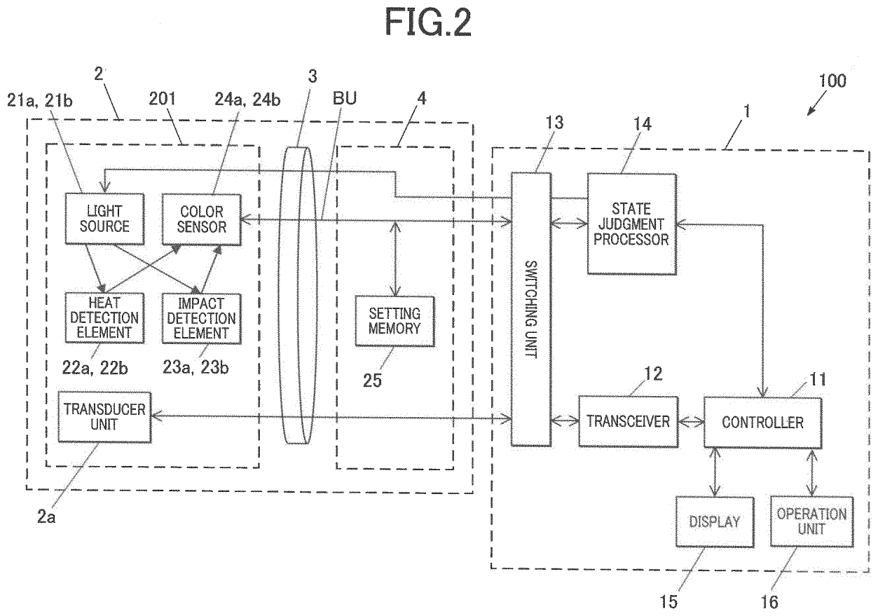

A first embodiment according to the present invention will be described with reference to FIG. 1 to FIG. 6. First, a device configuration of the present embodiment will be described with reference to FIG. 1 to FIG. 4. FIG. 1 is a schematic external view of an ultrasonic diagnosis device 100 of the present embodiment. FIG. 2 is a block diagram showing a functional configuration of the ultrasonic diagnosis device 100.

The ultrasonic diagnosis device 100 of the present embodiment is a device to perform ultrasonic diagnosis for use by operators such as a doctor and an engineer in a medical institution such as a hospital. As shown in FIG. 1 and FIG. 2, the ultrasonic diagnosis device 100 includes an ultrasonic probe 2 and an ultrasonic diagnosis device main body 1. The ultrasonic probe 2 transmits an ultrasonic wave (a transmission ultrasonic wave) to a subject such as an unshown patient's living body, and receives a reflected ultrasonic wave (an echo) of the ultrasonic wave reflected by this subject. The ultrasonic diagnosis device main body 1 is connected via a cable 3 and a connector 4 of the ultrasonic probe 2, transmits a transmission signal of an electric signal to a head section 201 of the ultrasonic probe 2, thereby causing the ultrasonic probe 2 to transmit a transmission ultrasonic wave to the subject, and images an internal state of the subject as an ultrasonic image on the basis of a reception signal that is the electric signal generated by the ultrasonic probe 2 in response to the reflected ultrasonic wave from the subject which is received by the ultrasonic probe 2.

The ultrasonic probe 2 includes a vibrator unit 2a including a plurality of vibrators of a piezoelectric element. In the vibrator unit 2a, for example, the plurality of vibrators are arranged in the form of a one-dimensional array in an azimuth direction (a scanning direction or an upward-downward direction). In the present embodiment, there is used the ultrasonic probe 2 including the vibrator unit 2a of n (e.g., 192) vibrators. Note that the vibrator unit 2a may be arranged in the form of a two-dimensional array.

As shown in FIG. 2, the ultrasonic probe 2 includes the head section 201, the cable 3 and the connector 4. The head section 201 includes the vibrator unit 2a, light sources 21a and 21b, heat detection elements 22a and 22b and impact detection elements 23a and 23b which constitute an abnormality detector, and color sensors 24a and 24b which constitute a state monitor. The connector 4 includes a setting memory 25. The head section 201 is stored in an exterior unit (an exterior cover, a grip or the like) which does not include any window members. The light source 21a, the heat detection element 22a, the impact detection element 23a and the color sensor 24a function as a set of monitors which has an identification number 1.

The light source 21a is a light emitting diode (LED) or a lamp, and emits light at a controlled power voltage from a state judgment processor 14 by control of a controller 11. The light source 21a irradiates the heat detection element 22a and the impact detection element 23a with light.

The heat detection element 22a is an element which changes in color in response to a surface temperature. The heat detection element 22a has, for example, a structure containing a temperature indicating material and sealed with a heat-resistant film. When heat changes from low to high, the color changes to green, yellow, and orange. The heat detection element 22a is selected that the color of the element does not overlap with the color of the impact detection element 23a.

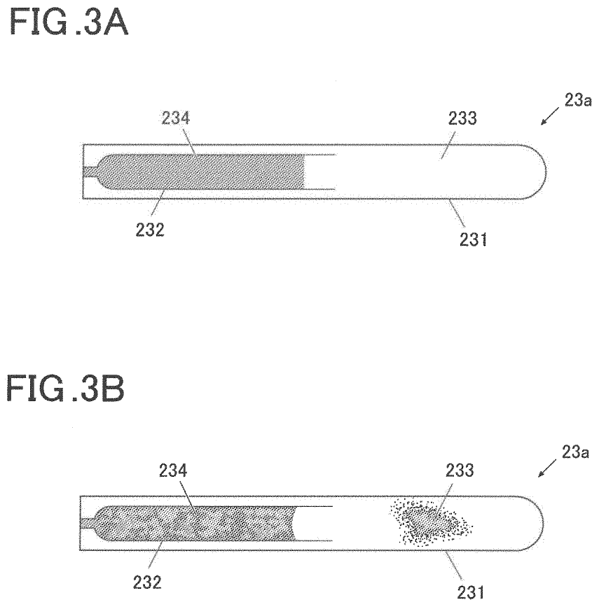

FIG. 3A is a schematic view of the impact detection element 23a before an impact is applied. FIG. 3B is a schematic view of the impact detection element 23a after the impact is applied.

The impact detection element 23a is an element which changes in color in response to the impact. As shown in FIG. 3A, the impact detection element 23a has, for example, a hermetically sealed double structure including an outer tube 231 of a glass tube and an inner tube 232 of a glass tube provided in the outer tube 231 and having one open end. In the outer tube 231, the inner tube 232 and a white diffusing agent 233 are enclosed. Colored ink 234 of a color other than white is enclosed in the inner tube 232. Here, colored ink 234 is red, but is not limited to this color.

When any impact is not applied to the colored ink 234, the ink is not diffused in the diffusing agent 233 due to surface tension. When the impact is applied, for example, from a side of the diffusing agent 233 to a side of the colored ink 234 in an axial direction, as shown in FIG. 3B, the surface tension is broken, the colored ink 234 is irreversibly diffused on the diffusing agent 233 side, and a red region increases. With elapse of time after the impact is applied, the red region enlarges due to the diffusion of the colored ink 234.

Furthermore, the impact detection element 23a has directivity. When the impact is applied from the colored ink 234 side to the diffusing agent 233 side in the axial direction, the colored ink 234 is hardly diffused. Consequently, the ultrasonic probe 2 has two impact detection elements 23a and 23b, and is disposed to reduce a direction in which the impact detection elements 23a and 23b cannot detect any impact. A plurality of impact detection elements 23a are prepared in accordance with a size of the impact to be detected, and the impact detection element to detect the impact that adversely affects the ultrasonic probe 2 is selected.

The impact detection element 23a is the irreversible detection element, and hence the element can detect not only an impact due to fall of the ultrasonic probe 2 operated by the operator during diagnosis or another operation or due to collision with another object but also an impact during storage of the ultrasonic probe 2 prior to use of the probe.

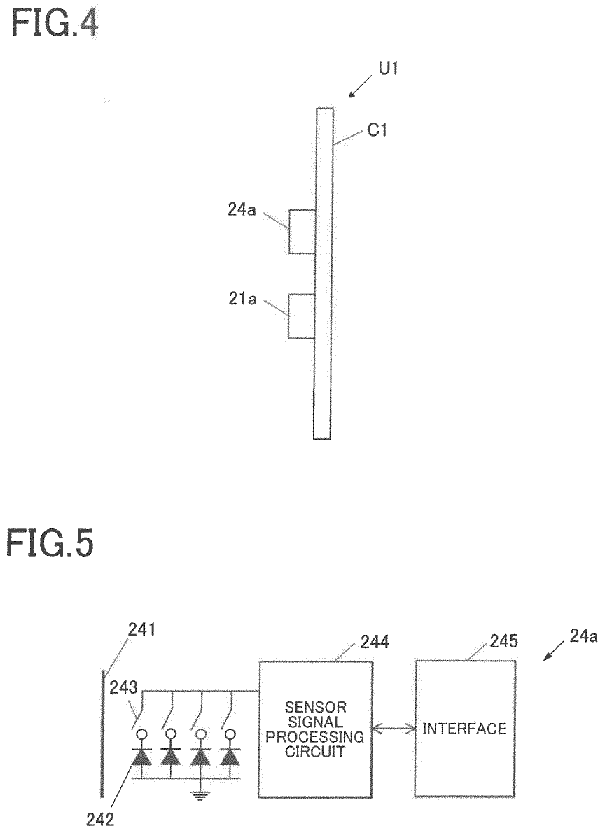

FIG. 4 is a view showing a sensor unit U1. FIG. 5 is a view showing a configuration of the color sensor 24a.

The color sensor 24a is a detector which detects an amount of light for each of wavelengths (e.g., red (R), blue (B), and infrared) and outputs light amount data for each wavelength to the state judgment processor 14. As shown in FIG. 4, the light source 21a and the color sensor 24a may constitute the sensor unit U1 mounted on a circuit substrate C1. An irradiation surface of the light source 21a and a detection surface of the color sensor 24a are directed to the heat detection element 22a and the impact detection element 23a. The heat detection element 22a and the impact detection element 23a which change to different colors are arranged in the vicinity, so that information of states of the heat detection element 22a and the impact detection element 23a can be acquired with one color sensor 24a.

As shown in FIG. 5, the color sensor 24a has, for example, a filter 241, light receivers 242, switches 243, a sensor signal processing circuit 244, and an interface 245.

The filter 241 is an infrared cut filter, and light emitted from the light source 21a and reflected by the heat detection element 22a and the impact detection element 23a is passed through the filter. The light receivers 242 are a plurality of photodiodes, and detect the light passed through the filter 241 and convert the light to an electric signal. The switches 243 are switches which switch on and off the photodiodes of the light receivers 242. The sensor signal processing circuit 244 is a circuit which calculates and outputs wavelength-specific light amount data from the electric signal input from the light receivers 242. The interface 245 is a hardware interface between the sensor signal processing circuit 244 and a bus BU.

The bus BU is a network connection bus connected between the ultrasonic probe 2 (the color sensors 24a and 24b) and the ultrasonic diagnosis device main body 1 (the state judgment processor 14 via a switching unit 13), and is a bus of a serial communication system, such as RS232, RS422/RS485, I2C, or SPI. The bus BU may be a bus of another communication system for network connection, such as Ethernet (a registered trademark) or a parallel communication system. By use of the bus BU, the ultrasonic probe 2 can perform diagnosis by specifying an abnormal state and a region where the state occur, without increasing signal lines.

The setting memory 25 is a non-volatile readable and writable storage such as a flash memory connected to the bus BU. The setting memory 25 stores each setting data as information to judge whether or not the wavelength-specific light amount data indicates (occurrence of) the impact, indicates (occurrence of) a heating abnormality, or indicates (occurrence of) a heating caution. The heating caution indicates that any heating abnormality does not occur but heat is so high that caution is required. The setting data is data including a type of ultrasonic probe 2 and corresponding to identification information of the heat detection elements 22a and 22b and the impact detection elements 23a and 23b which constitute an abnormality detector.

For example, the setting data has information in a range from a value R1 to a value R2 in which the light amount data of red indicates the impact, information in a range from a value G1 to a value G2 in which the light amount data of green indicates the impact, and information in a range from a value B1 to a value B2 in which the light amount data of blue indicates the impact. Furthermore, the setting data has information in a range from a value R3 to a value R4 in which the light amount data of red indicates the heating abnormality, information in a range from a value G3 to a value G4 in which the light amount data of green indicates the heating abnormality, and information in a range from a value B3 to a value B4 in which the light amount data of blue indicates the heating abnormality. Additionally, the setting data has information in a range from a value R5 to a value R6 in which the light amount data of red indicates the heating caution, information in a range from a value G5 to a value G6 in which the light amount data of green indicates the heating caution, and information in a range from a value B5 to a value B6 in which the light amount data of blue indicates the heating caution.

The connector 4 is connected to the head section 201 via the cable 3, and removably and electrically connected to the ultrasonic diagnosis device main body 1.

The light source 21b, the heat detection element 22b, the impact detection element 23b and the color sensor 24b function as a set of monitors which has an identification number 2, and are similar to the light source 21a, the heat detection element 22a, the impact detection element 23a and the color sensor 24a, respectively. Consequently, description is omitted.

As shown in FIG. 2, the ultrasonic diagnosis device main body 1 includes, for example, the controller 11 as an image former, a transceiver 12, a switching unit 13, the state judgment processor 14 as a judging unit, a display 15, and an operation unit 16.

The controller 11 includes, for example, a central processing unit (CPU), a read only memory (ROM), and a random access memory (RAM). The controller 11 reads various processing programs including a system program stored in the ROM and the like to develop the programs in the RAM, and controls respective components of the ultrasonic diagnosis device 100 in accordance with the developed programs. In the ROM, there are stored the program to form ultrasonic image data, and a first abnormality detection program to execute first abnormality detection processing which will be described later.

The transceiver 12 is a circuit which, in accordance with the control of the controller 11, supplies the transmission signal as the electric signal to the head section 201 via the connector 4 and the cable 3 and generates a transmission ultrasonic wave.

Furthermore, the transceiver 12, in accordance with the control of the controller 11, receives the reception signal as an electric signal from the head section 201 via the cable 3 and the connector 4 of the ultrasonic probe 2.

The controller 11 also functions as the image former of the ultrasonic image, and an image display controller.

Furthermore, the controller 11 performs coordinate transformation or the like on B-mode image data of a frame to convert the data to an image signal, and outputs the signal to the display 15.

The switching unit 13 has a plurality of ports connected to the signal line and a power line of the connector 4 of the ultrasonic probe 2 and, in accordance with the control of the controller 11, opens and closes the port to turn on/off passage of the signal and power.

The state judgment processor 14, in accordance with the control of the controller 11, drives the light sources 21a and 21b and the color sensors 24a and 24b, judges whether or not the wavelength-specific light amount data input from the color sensors 24a and 24b indicates a state of abnormality (the impact, the heating abnormality or the heating caution), by use of the setting data read from the setting memory 25, and stores abnormality information in the setting memory 25.

As the display 15, there can be applied a display such as a liquid crystal display (LCD), a cathode ray tube (CRT) display, an organic electronic luminescence (EL) display, an inorganic EL display, or a plasma display. The display 15 displays the image signal of the ultrasonic image input from the controller 11, or various types of display information.

The operation unit 16 includes, for example, various switches, a ball, a track ball, a mouse, and a keyboard to input data such as a command instructing start of the diagnosis, and personal information of a subject, and outputs an operation signal to the controller 11. The operation unit 16 is provided on a display screen of the display 15, and may include a touch panel which accepts user's touch input.

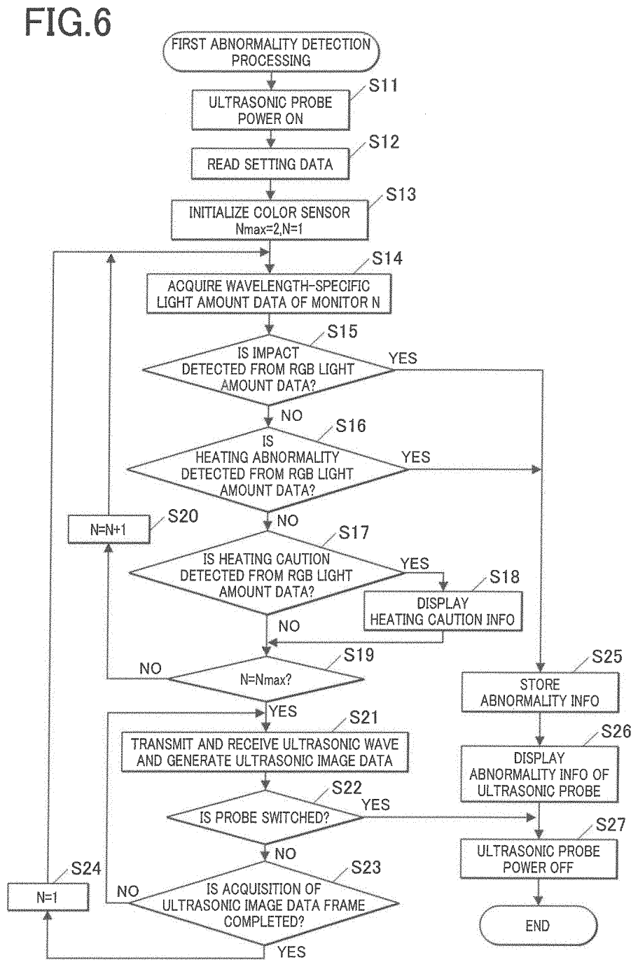

Next, an operation of the ultrasonic diagnosis device 100 will be described with reference to FIG. 6. FIG. 6 is a flowchart showing the first abnormality detection processing.

In the ultrasonic diagnosis device 100, for example, upon receiving a trigger generated by inputting an instruction for generation and display of the ultrasonic image via the operation unit 16 by the operator and connecting the ultrasonic probe 2 to the ultrasonic diagnosis device main body 1, the controller 11 executes the first abnormality detection processing in accordance with the first abnormality detection program stored in the ROM. Here, a subject of the execution of the first abnormality detection processing is the controller 11, but in steps in which the controller 11 controls and operates the state judgment processor 14, the state judgment processor 14 is described as the subject.

As shown in FIG. 6, the controller 11 first opens the ports of the signal line and the power line of the switching unit 13, to allow transmission and reception of the signal to and from the ultrasonic probe 2 connected to the ultrasonic diagnosis device main body 1, and the controller starts supply of a power voltage to the ultrasonic probe 2 to turn on the probe (step S11). Then, the state judgment processor 14 reads, from the setting memory 25, the setting data including the type of connected ultrasonic probe 2 and corresponding to the identification information of the heat detection elements 22a and 22b and the impact detection elements 23a and 23b which constitute the abnormality detector (step S12). Then, the state judgment processor 14 initializes the color sensors 24a and 24b, and the controller 11 substitutes 2 for identification number Nmax of the monitor, and substitutes 1 for variable N of the identification number of the monitor of the ultrasonic probe 2 (step S13).

Then, the state judgment processor 14 drives a monitor N and acquires the wavelength-specific light amount data output from the color sensor of the monitor N (step S14). Then, the state judgment processor 14 judges whether or not the wavelength-specific (R, G or B) light amount data acquired in the step S14 is in the range of the setting data of each of R, G and B, by use of the setting data for impact detection which is read in the step S12, to judge whether or not the impact is detected (step S15). In the step S15, for example, when the light amount data of R is in the range from the value R1 to the value R2, the light amount data of G is in the range from the value G1 to the value G2 and the light amount data of B is in the range from the value B1 to the value B2, it is judged that the impact is present.

When any impact is not present (the step S15; NO), the state judgment processor 14 judges whether or not the wavelength-specific (R, G, B) light amount data acquired in the step S14 is in the range of the setting data of R, G or B, by use of the setting data for heating abnormality detection which is read in the step S12, to judge whether or not the heating abnormality is detected (step S16). In the step S16, for example, when the light amount data of R is in the range from the value R3 to the value R4, the light amount data of G is in the range from the value G3 to the value G4 and the light amount data of B is in the range from the value B3 to the value B4, it is judged that the heating abnormality is present.

When any heating abnormality is not present (the step S16; NO), the state judgment processor 14 judges whether or not the wavelength-specific (R, G or B) light amount data acquired in the step S14 is in the range of the setting data of R, G or B, by use of the setting data for heating caution detection which is read in the step S12, to judge whether or not the heating caution is detected (step S17). In the step S17, for example, when the light amount data of R is in the range from the value R5 to the value R6, the light amount data of G is in the range from the value G5 to the value G6 and the light amount data of B is in the range from the value B5 to the value B6, it is judged that the heating caution is present.

When the heating caution is present (the step S17; YES), the controller 11 displays, in the display 15, heating caution information which alerts the heating caution of the ultrasonic probe 2 (step S18). After the step S18 is executed or when any heating caution is not present (the step S17; NO), the controller 11 judges whether or not the variable N=Nmax (here Nmax=2) (step S19). When N=1 (the step S19; NO), the controller 11 increments the variable N (step S20), and shifts to the step S14.

When N=Nmax (the step S19; YES), the controller 11 controls the transceiver 12 so that the ultrasonic wave is transmitted from the vibrator unit 2a, sound ray data is generated from the reception signal of the reflected ultrasonic wave which is received, and the ultrasonic image data is generated from the sound ray data (step S21). Then, the controller 11 judges whether or not switching of the ultrasonic probe 2 is input from the operator via the operation unit 16 (step S22).

When any input of the switching of the ultrasonic probe 2 is not present (the step S22; NO), the controller 11 judges whether or not acquisition of one frame of the ultrasonic image data generated in the step S21 is completed (step S23). When the acquisition of the one frame of the ultrasonic image data is not completed (the step S23; NO), the controller shifts to the step S21. When the acquisition of the one frame of the ultrasonic image data is completed (the step S23; YES), the controller 11 substitutes 1 for the variable N, and shifts to the step S14. The one frame of the generated ultrasonic image data is displayed, for example, as one frame of ultrasonic image in live display of the ultrasonic image in the display 15.

When the impact is present (the step S15; YES) or when the heating abnormality is present (the step S16; YES), the state judgment processor 14 associates the wavelength-specific light amount data acquired in the step S14 with the type of abnormality (the impact or the heating abnormality) and the current variable N (N=1: the heat detection element 22a or the impact detection element 23a, or N=2: the heat detection element 22b or the impact detection element 23b), to store the data in the setting memory 25 (step S25).

Then, the controller 11 displays the information used in the step S25, including the type of abnormality and the current variable N, and indicating the abnormality, in the display 15 (step S26). After the step S26 is executed or when the input of the switching of the ultrasonic probe 2 is present (the step S22; YES), the controller 11 stops the transmission and reception of the signal to and from the connected ultrasonic probe 2, stops the supply of the power voltage to the ultrasonic probe 2 to turn off the probe, and closes the ports of the signal line and power line of the switching unit 13 (step S27), thereby ending the first abnormality detection processing.

As described above, according to the present embodiment, the ultrasonic probe 2 includes the vibrator unit 2a which transmits the ultrasonic wave in response to the transmission signal and generates the reception signal of the reflected ultrasonic wave, the heat detection elements 22a and 22b and the impact detection elements 23a and 23b which are provided in the ultrasonic probe 2, detect the abnormality and indicate the abnormality by a color, and the color sensors 24a and 24b which are provided in the ultrasonic probe 2, monitor the states of the heat detection elements 22a and 22b and the impact detection elements 23a and 23b by the color, and output the monitoring information.

Consequently, there can be provided the ultrasonic probe 2 and the ultrasonic diagnosis device 100 which have high safety and high reliability and in which the internal state of the ultrasonic probe 2 can be monitored and the abnormality of the ultrasonic probe 2 can be easily and securely detected in accordance with the color, without opening the exterior cover of the ultrasonic probe 2 and without need to provide any window member in the exterior cover, so that color change of the window member due to disinfection does not occur.

Furthermore, in the impact detection element 23a or 23b, the colored ink is diffused in response to the impact so that monitoring is possible. Consequently, there can be provided the ultrasonic probe 2 and the ultrasonic diagnosis device 100 in which the impact resulting from the fall, the collision or the like can be securely detected by the color and which have high safety and high reliability.

Additionally, the color sensors 24a and 24b monitor the heat detection elements 22a and 22b and the impact detection elements 23a and 23b and output the wavelength-specific light amount data. Consequently, the internal state of the ultrasonic probe 2 can be monitored in accordance with a wavelength-specific light amount, and the change of the internal state can be reliably grasped.

In addition, the heat detection elements 22a and 22b change in color in response to a surface temperature based on heat. Consequently, there can be provided the ultrasonic probe 2 and the ultrasonic diagnosis device 100 in which abnormal heating can be securely detected by the color and which have high safety and high reliability.

Furthermore, the impact detection elements 23a and 23b constitute an irreversible abnormality detector which holds a detected state upon detecting the impact. Consequently, also in a state where power is off, for example, during the storage of the ultrasonic probe 2, the probe changes the state upon receiving the impact, and can hold the changed state, and when the power of the ultrasonic probe 2 is turned on, the impact received in the state where the power is off can be confirmed. Additionally, the heat detection elements 22a and 22b may constitute an irreversible abnormality detector which holds the detected state upon detecting heat.

Additionally, the color sensor 24a (24b) simultaneously monitors the heat detection element 22a (22b) and the impact detection element 23a (23b) as a plurality of types of abnormality detectors. Consequently, one color sensor can simultaneously detect a plurality of types of abnormalities (the impact and the heating).

In addition, the heat detection element 22a (22b) and the impact detection element 23a (23b) as the plurality of types of abnormality detectors indicate the detection of the abnormalities in mutually different colors.

Consequently, there can be provided the ultrasonic probe 2 and the ultrasonic diagnosis device 100 in which one color sensor can simultaneously and accurately detect a plurality of types of abnormalities (the impact and the heating) and which have high reliability.

Furthermore, the ultrasonic probe 2 includes the setting memory 25 to store the setting data which corresponds to the identification information of the ultrasonic probe 2 and which is threshold information for use in judgment of the abnormality from the wavelength-specific light amount data. Consequently, the color sensors 24a and 24b, the heat detection elements 22a and 22b and the impact detection elements 23a and 23b individually differ in initial state and installation position, but the setting data for the judgment is provided on an ultrasonic probe 2 side, so that individual variations are corrected and the reliability of the judgment improves.

Additionally, the ultrasonic diagnosis device 100 includes the ultrasonic probe 2, the transceiver 12 which inputs the transmission signal into the vibrator unit 2a and acquires the reception signal, the controller 11 which forms the ultrasonic image data from the input reception signal, and the state judgment processor 14 which judges the abnormality of the ultrasonic probe 2 from the setting data stored in the setting memory 25 and the wavelength-specific light amount data output from the color sensors 24a and 24b. When it is judged that the abnormality is present, the controller 11 stops the operation of the ultrasonic probe 2. Consequently, there can be provided the ultrasonic diagnosis device 100 which prevents the ultrasonic probe 2 from being used when it is judged that the abnormality is present, to improve safety.

Furthermore, when it is judged that the abnormality is present, the controller 11 stops the transmission and reception of the signal to and from the ultrasonic probe 2 and the power supply to the ultrasonic probe 2. Consequently, there can be provided the ultrasonic diagnosis device 100 which prevents the ultrasonic probe 2 from being used when it is judged that the abnormality is present, so that safety can further improve and power consumption can be decreased.

Additionally, when it is judged that the abnormality is present, the controller 11 stores, in the setting memory 25, the abnormality information including the wavelength-specific light amount data corresponding to the abnormality. Consequently, there can be provided the ultrasonic probe 2 and the ultrasonic diagnosis device 100 in which a cause for the abnormality can be easily identified, when a repairer reads and confirms the abnormality information from the setting memory 25 during repair or inspection of the ultrasonic probe 2.

In addition, when it is judged that the abnormality is present, the controller 11 displays the abnormality information indicating the abnormality in the display 15. Consequently, there can be provided the ultrasonic probe 2 and the ultrasonic diagnosis device 100 in which the abnormality can be easily identified, when the repairer visually confirms the abnormality information.

Furthermore, the state judgment processor 14 and the color sensors 24a and 24b are connected to the bus BU as a network. Thus, the plurality of color sensors are connected to the network, so that required information can be set and acquired from the color sensors without increasing lines which connect the ultrasonic probe 2 and a main body side of the ultrasonic diagnosis device.

Additionally, when the state judgment processor 14 is connected to the ultrasonic probe 2, the processor automatically judges the abnormality of the ultrasonic probe 2 from the wavelength-specific light amount data output from the color sensors 24a and 24b. Consequently, self-diagnosis of the ultrasonic probe 2 can be performed prior to use of the probe, and the ultrasonic probe 2 and the ultrasonic diagnosis device 100 which have high safety and high reliability can be provided.

### Second Embodiment

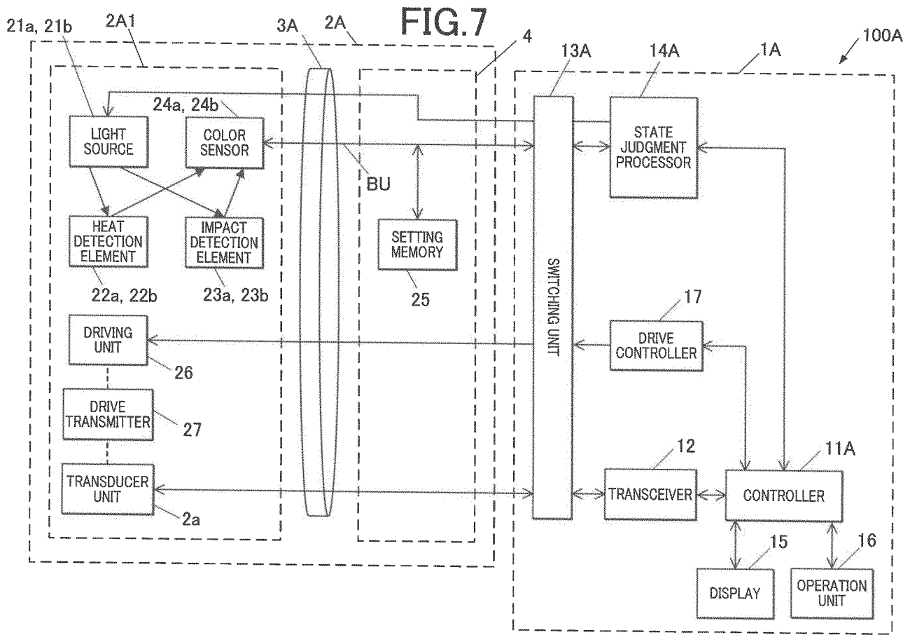

A second embodiment according to the present invention will be described with reference to FIG. 7 to FIG. 9B. FIG. 7 is a block diagram showing a functional configuration of an ultrasonic diagnosis device 100A. FIG.

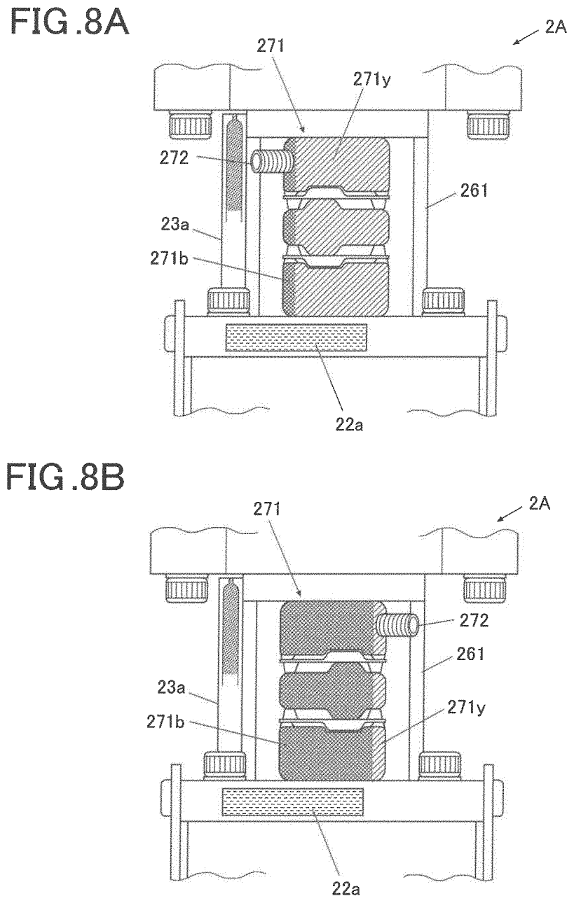

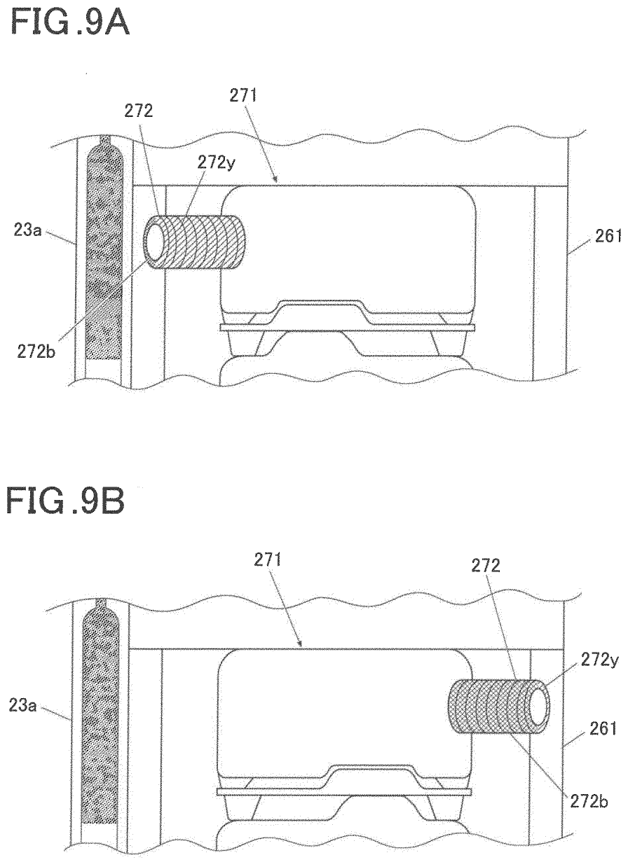

8A is a view showing an ultrasonic probe 2A having a coupling 271 at a left rotation position. FIG. 8B is a view showing the ultrasonic probe 2A having the coupling 271 at a right rotation position. FIG. 9A is a view showing a pin 272 at the left rotation position. FIG. 9B is a view showing the pin 272 at the right rotation position.

In the present embodiment, the ultrasonic diagnosis device 100A shown in FIG. 7 is used in place of the ultrasonic diagnosis device 100 of the first embodiment. In the ultrasonic diagnosis device 100A, components similar to the components of the ultrasonic diagnosis device 100 are denoted with the same reference signs and description of the components is omitted.

The ultrasonic diagnosis device 100A includes the ultrasonic probe 2A and an ultrasonic diagnosis device main body 1A. The ultrasonic probe 2A causes a vibrator unit 2a to mechanically swing right and left in a direction perpendicular to a scanning direction, to transmit and receive an ultrasonic wave and to obtain three-dimensional ultrasonic image data.

The ultrasonic probe 2A includes a head section 2A1, a cable 3A, and a connector 4. The head section 2A1 includes the vibrator unit 2a, light sources 21a and 21b, heat detection elements 22a and 22b, impact detection elements 23a and 23b, color sensors 24a and 24b, a driving unit 26, and a drive transmitter 27. The connector 4 includes a setting memory 25 as a first or second storage.

The driving unit 26 includes a motor and others and, in accordance with control of a drive controller 17, rotates, drives and swings the vibrator unit 2a, for example, in the direction perpendicular to the scanning direction via the drive transmitter 27. The drive transmitter 27 includes a coupling, a pulley and other components, and transmits the rotation driving of the driving unit 26 to the vibrator unit 2a.

The coupling and other components which constitute the drive transmitter 27 are painted with a paint in a predetermined color, so that a driving position of each component can be detected with the color sensors 24a and 24b. Consequently, the light source 21a and the color sensor 24a are directed not only to the heat detection element 22a and the impact detection element 23a but also to the drive transmitter 27. Furthermore, an irradiation surface of the light source 21b and a detection surface of the color sensor 24b are directed not only to the heat detection element 22b and the impact detection element 23b but also to the drive transmitter 27.

For example, as shown in FIG. 8A, the ultrasonic probe 2A includes the drive transmitter 27, the heat detection element 22a, the impact detection element 23a, a frame 261 and the like. The frame 261 is provided between a member on a side of the driving unit 26 and a member on a side of the vibrator unit 2a, to fix the drive transmitter 27 to the frame. The drive transmitter 27 has the coupling 271 which transmits the rotation driving, and the pin 272 provided in the coupling 271. The coupling 271 has a yellow region portion 271y painted with a yellow paint, and a blue region portion 271b painted with a blue paint. As shown in FIG. 8A, when the coupling 271 is rotated to the left side and the vibrator unit 2a is positioned at a left end, the yellow region portion 271y looks larger than the blue region portion 271b. Furthermore, more yellow components are detected in wavelength-specific light amount data by the color sensor 24a. Alternatively, as shown in FIG. 8B, when the coupling 271 is rotated to the right side and the vibrator unit 2a is also positioned at a right end, the blue region portion 271b looks larger than the yellow region portion 271y. Furthermore, more blue components are detected in the wavelength-specific light amount data by the color sensor 24a. In consequence, the yellow region portion 271y and the blue region portion 271b are painted with the paints.

Alternatively, as shown in FIG. 9A, the drive transmitter 27 may have the coupling 271 and the pin 272, and the pin 272 may be painted with a paint in place of the coupling 271. The pin 272 has a yellow region portion 272y painted with the yellow paint, and a blue region portion 272b painted with the blue paint. As shown in FIG. 9A, when the pin 272 is rotated to the left side and the vibrator unit 2a is also positioned at the left end, the yellow region portion 272y looks larger than the blue region portion 272b, and more yellow components are detected in the wavelength-specific light amount data by the color sensor 24a. Alternatively, as shown in FIG. 9B, when the pin 272 is rotated to the right side and the vibrator unit 2a is also positioned at the right end, the blue region portion 271b looks larger than the yellow region portion 271y. More blue components are also detected in the wavelength-specific light amount data by the color sensor 24a. In consequence, the yellow region portion 272y and the blue region portion 272b are painted with the paints.

Targets to be painted in color are not limited to the coupling 271 and the pin 272 of the drive transmitter 27, and may be other driving components of the driving unit 26 and the drive transmitter 27, for example, the pulley, a belt and a rotary shaft. Furthermore, types and the number of color of the paint are not limited to two colors of yellow and blue. Note that it is preferable that the color of the paint is different from a color shown by the heat detection element 22a or 22b or the impact detection element 23a or 23b. Additionally, the number of types of the colors of the paint is not limited to two.

As shown in FIG. 7, the cable 3A further has a signal line and a power line which extend from the drive controller 17 to the driving unit 26 in addition to the signal line of the cable 3.

The ultrasonic diagnosis device main body 1A includes a controller 11A, a transceiver 12, a switching unit 13A, a state judgment processor 14A, a display 15, an operation unit 16, and the drive controller 17. The controller 11A is similar to the controller 11 of FIG. 2, but causes the state judgment processor 14A to detect a drive position of the vibrator unit 2a. The switching unit 13A opens and closes a port of the signal line which extends from the drive controller 17 to the driving unit 26, in addition to the port of the switching unit 13 of FIG. 2.

The state judgment processor 14A, in accordance with the control of the controller 11A, drives the light sources 21a and 21b and the color sensors 24a and 24b, judges whether or not the wavelength-specific light amount data input from the color sensor 24a or 24b indicates a state of abnormality (an impact, a heating abnormality or a heating caution), by use of setting data read from the setting memory 25, also judges a driven position of a position of the vibrator unit 2a (the drive transmitter 27), and stores abnormality information in the setting memory 25.

The drive controller 17 drives the driving unit 26 as much as an optional amount in accordance with the control of the controller 11A. Note that the ultrasonic probe 2A may include an encoder which detects and encodes a drive amount of the drive transmitter 27. The encoder feeds encoded drive amount data back to the drive controller 17. The drive controller 17 executes feedback control of the driving unit 26 in accordance with the input drive amount data.

The setting memory 25 stores the setting data including a type of ultrasonic probe 2A and corresponding to identification information of the heat detection elements 22a and 22b and the impact detection elements 23a and 23b which constitute an abnormality detector, the setting data being for use in judging the impact, the heating abnormality and the heating caution. The setting memory additionally stores setting data which corresponds to, for example, light amounts of yellow and blue in the wavelength-specific light amount data and which is positional information of the drive transmitter 27 (the coupling 271 or the pin 272).

Next, an operation of the ultrasonic diagnosis device 100A will be described. In the ultrasonic diagnosis device 100A, the controller 11A executes processing similar to the first abnormality detection processing shown in FIG. 6. Only processing that is different from the first abnormality detection processing will be described.

In step S25, the state judgment processor 14A detects the positional information of the drive transmitter 27 from the wavelength-specific light amount data acquired in step S14 by use of the setting data of the positional information of the drive transmitter 27 which is read in step S12, and generates the positional information of the vibrator unit 2a which corresponds to the positional information of the drive transmitter 27. Then, the state judgment processor 14A associates the wavelength-specific light amount data acquired in the step S14 with a type of abnormality, current variable N and the generated positional information of the vibrator unit 2a, to store the data in the setting memory 25.

As described above, according to the present embodiment, the ultrasonic probe 2A includes the driving unit 26 and the drive transmitter 27 which drive the vibrator unit 2a and transmit the driving. The drive transmitter 27 shows different colors in accordance with drive states. The color sensors 24a and 24b monitor the heat detection elements 22a and 22b, the impact detection elements 23a and 23b and the drive transmitter 27, and outputs the wavelength-specific light amount data. Consequently, there can be provided the ultrasonic probe 2A and the ultrasonic diagnosis device 100A in which the drive state can be monitored simultaneously with the abnormality state, the drive state during the abnormality can be detected, a drive abnormality can be easily detected, drive can be easily inspected, and high reliability can be provided.

### Third Embodiment

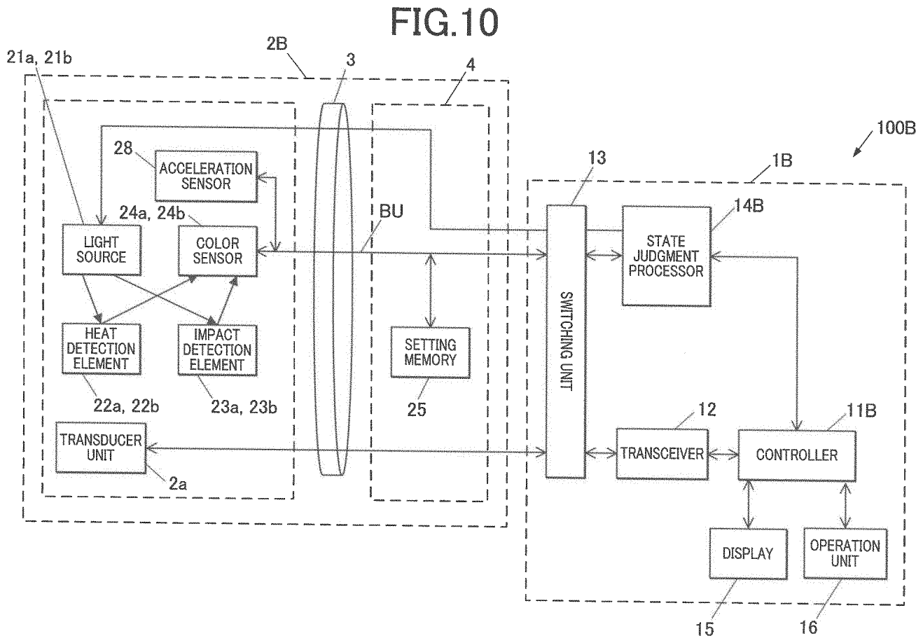

A third embodiment according to the present invention will be described with reference to FIG. 10. FIG. 10 is a block diagram showing a functional configuration of an ultrasonic diagnosis device 100B.

In the present embodiment, the ultrasonic diagnosis device 100B shown in FIG. 10 is used in place of the ultrasonic diagnosis device 100 of the first embodiment. In the ultrasonic diagnosis device 100B, components similar to the components of the ultrasonic diagnosis device 100 are denoted with the same reference signs and description of the components is omitted.

The ultrasonic diagnosis device 100B includes an ultrasonic probe 2B and an ultrasonic diagnosis device main body 1B. The ultrasonic probe 2B includes a head section 2B1, a cable 3 and a connector 4. The head section 2B1 includes a vibrator unit 2a, light sources 21a and 21b, heat detection elements 22a and 22b, impact detection elements 23a and 23b, color sensors 24a and 24b, and an acceleration sensor 28 as an acceleration detector.

The acceleration sensor 28 is the detector connected to a bus BU, for example, to detect accelerations in respective directions of three axes in a three-dimensional space and to output the accelerations as acceleration data of the respective axes to a state judgment processor 14B.

The ultrasonic diagnosis device main body 1B includes a controller 11B, a transceiver 12, a switching unit 13, the state judgment processor 14B, a display 15, and an operation unit 16. The controller 11B is similar to the controller 11 of FIG. 2, but causes the state judgment processor 14B to detect the accelerations.

The state judgment processor 14B, in accordance with control of the controller 11B, drives the light sources 21a and 21b and the color sensors 24a and 24b, and judges whether or not wavelength-specific light amount data input from the color sensors 24a and 24b indicates a state of abnormality (an impact, a heating abnormality or a heating caution), by use of setting data read from a setting memory 25. Furthermore, the state judgment processor drives the acceleration sensor 28, acquires the acceleration data, and stores the abnormality information in the setting memory 25.

Next, an operation of the ultrasonic diagnosis device 100B will be described. In the ultrasonic diagnosis device 100B, the controller 11B executes processing similar to the first abnormality detection processing shown in FIG. 6, and therefore only processing different from the first abnormality detection processing will be described.

In step S14, the state judgment processor 14B drives a monitor N to acquire the wavelength-specific light amount data output from the color sensor of the monitor N, and drives the acceleration sensor 28 to acquire the acceleration data output from the acceleration sensor 28.

In step S25, the state judgment processor 14B associates the wavelength-specific light amount data acquired in the step S14 with a type of abnormality, current variable N and the acceleration data acquired in the step S14, to store the data in the setting memory 25.

As described above, according to the present embodiment, the ultrasonic probe 2B includes the acceleration sensor 28 which detects the acceleration of the ultrasonic probe 2B. Consequently, there can be provided the ultrasonic probe 2B and the ultrasonic diagnosis device 100B in which an acceleration state is monitored simultaneously with an abnormality state, an abnormal acceleration state due to fall of the ultrasonic probe 2B, an impact from outside, or the like can be detected, and high reliability can be provided.

Note that the impact may be judged by using the wavelength-specific light amount data and the acceleration data. For example, it is assumed that a threshold value of the acceleration data indicating the impact is stored as the setting data in the setting memory 25. In step S15 of the first abnormality detection processing, the state judgment processor 14B may judge whether or not each wavelength-specific (R, G, or B) light amount data acquired in the step S14 is within a range of the setting data for R, G, or B, by use of the setting data of the acceleration for impact detection which is read in step S12. Furthermore, the state judgment processor may judge whether or not the acceleration data acquired in the step S14 is in excess of a threshold value, by use of the setting data of the acceleration for the impact detection, and may, on a basis of this judgment, judge whether or not the impact is detected. According to this configuration, the impact can be detected in real time, and the reliability of the impact detection can further improve.

### Fourth Embodiment

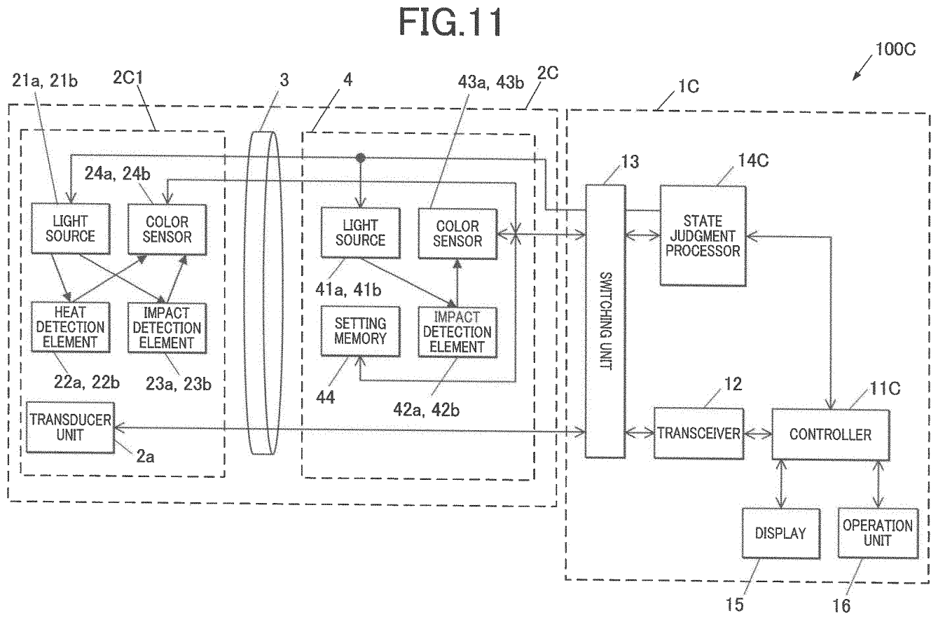

A fourth embodiment according to the present invention will be described with reference to FIG. 11. FIG. 11 is a block diagram showing a functional configuration of an ultrasonic diagnosis device 100C.

In the present embodiment, the ultrasonic diagnosis device 100C shown in FIG. 11 is used in place of the ultrasonic diagnosis device 100 of the first embodiment. In the ultrasonic diagnosis device 100C, components similar to the components of the ultrasonic diagnosis device 100 are denoted with the same reference signs and description of the components is omitted.

The ultrasonic diagnosis device 100C includes an ultrasonic probe 2C and an ultrasonic diagnosis device main body 1C. The ultrasonic probe 2C includes a head section 2C1, a cable 3 and a connector 4. The head section 2C1 includes a vibrator unit 2a, light sources 21a and 21b, heat detection elements 22a and 22b, impact detection elements 23a and 23b, and color sensors 24a and 24b.

The connector 4 is a connector which is connected to the head section 2C1 via the cable 3, and detachably and electrically connected to the ultrasonic diagnosis device main body 1C. The connector 4 includes light sources 41a and 41b, impact detection elements 42a and 42b which constitute an abnormality detector, color sensors 43a and 43b which constitute a state monitor, and a setting memory 44 as a first or second storage. Note that the connector 4 may include heat detection elements.

The light sources 41a and 41b, the impact detection elements 42a and 42b and the color sensors 43a and 43b are similar to the light sources 21a and 21b, the impact detection elements 23a and 23b, and the color sensors 24a and 24b, respectively. It is considered that the light source 41a, the impact detection element 42a and the color sensor 43a constitute a set of monitors which has an identification number 3. It is considered that the light source 41b, the impact detection element 42b and the color sensor 43b constitute a set of monitors which has an identification number 4.

The setting memory 44 has a configuration similar to the configuration of the setting memory 25, and stores setting data including a type of ultrasonic probe 2C and corresponding to identification information of the heat detection elements 22a and 22b and the impact detection elements 23a, 23b, 42a and 42b which constitute the abnormality detector. As the setting data, there is stored each setting data as information to judge whether or not wavelength-specific light amount data indicates (occurrence of) an impact, indicates (occurrence of) a heating abnormality, or indicates (occurrence of) a heating caution. The heating caution indicates that any heating abnormality is not present, but heat is so high that the caution is required. The color sensors 43a and 43b and the setting memory 44 are connected to a bus BU.

The ultrasonic diagnosis device main body 1C includes a controller 11C, a transceiver 12, a switching unit 13, a state judgment processor 14C, a display 15, and an operation unit 16. The controller 11C is similar to the controller 11 of FIG. 2 except that the controller causes the state judgment processor 14C to drive the light sources 21a, 21b, 41a and 41b and the color sensors 24a, 24b, 43a and 43b, to acquire the wavelength-specific light amount data, to judge the abnormality, and to store abnormality information in the setting memory 44.

The state judgment processor 14C, in accordance with control of the controller 11C, drives the light sources 21a, 21b, 41a and 41b and the color sensors 24a, 24b, 43a and 43b, judges whether or not the wavelength-specific light amount data input from the color sensors 24a, 24b, 43a and 43b indicates a state of an abnormality (the impact, the heating abnormality or the heating caution) by use of the setting data read from the setting memory 44, and stores the abnormality information in the setting memory 44.

Next, an operation of the ultrasonic diagnosis device 100C will be described. In the ultrasonic diagnosis device 100C, the controller 11C executes processing similar to the first abnormality detection processing shown in FIG. 6, and therefore only processing different from the first abnormality detection processing will be described.

In the present embodiment, identification number Nmax is 4, and hence variable N takes values from 1 to 4. Consequently, in step S13, the controller 11C substitutes 4 for the identification number Nmax of the monitor, and in step S19, the controller 11C judges whether or not the variable N=Nmax (here, Nmax=4). In step S25, the state judgment processor 14 associates the wavelength-specific light amount data acquired in step S14 with a type of abnormality (the impact or the heating abnormality), and the current variable N (N=1: the heat detection element 22a or the impact detection element 23a, N=2: the heat detection element 22b or the impact detection element 23b, N=3: the impact detection element 42a, or N=4: the impact detection element 42b), and the processor stores the data in the setting memory 44.

As described above, according to the present embodiment, the ultrasonic probe 2C includes the head section 2C1 having the vibrator unit 2a, the cable 3 connected to the head section 2C1, and the connector 4 connected to the cable 3 and connected to (the state judgment processor 14C of) the ultrasonic diagnosis device main body 1C of the ultrasonic diagnosis device 100C. The head section 2C1 and the connector 4 have (the heat detection elements,) the impact detection elements and the color sensors, respectively. Consequently, if the impact or the like is applied to a side of the head section 2C1 or a side of the connector 4 during conveyance, during attaching/detaching to/from the ultrasonic diagnosis device main body 1C, or during use, the side might fail. To solve such problems, there can be provided the ultrasonic probe 2C and the ultrasonic diagnosis device 100C in which the abnormalities of both the head section 2C1 side and the connector 4 side are detected and high reliability can be provided.

### Fifth Embodiment

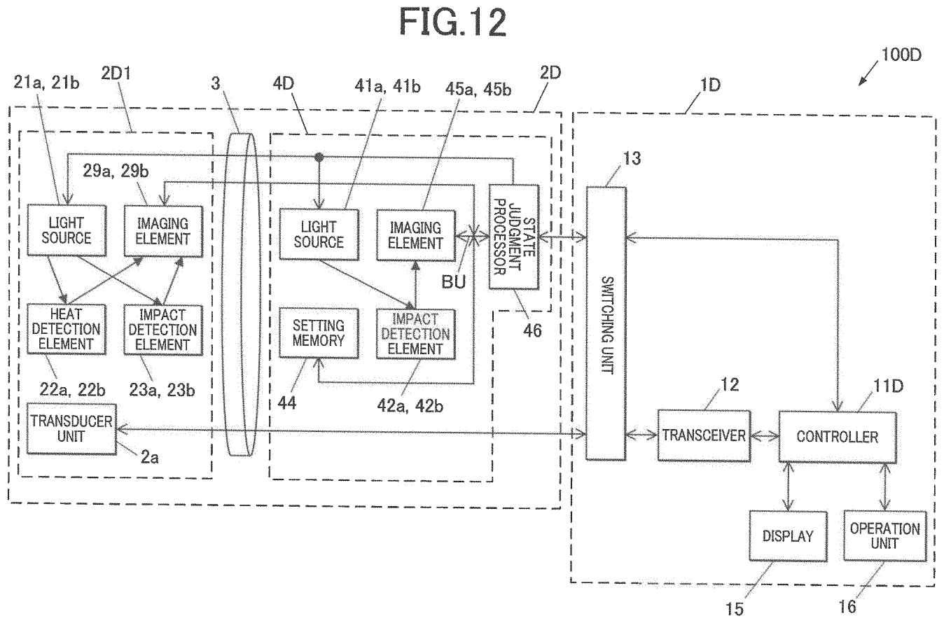

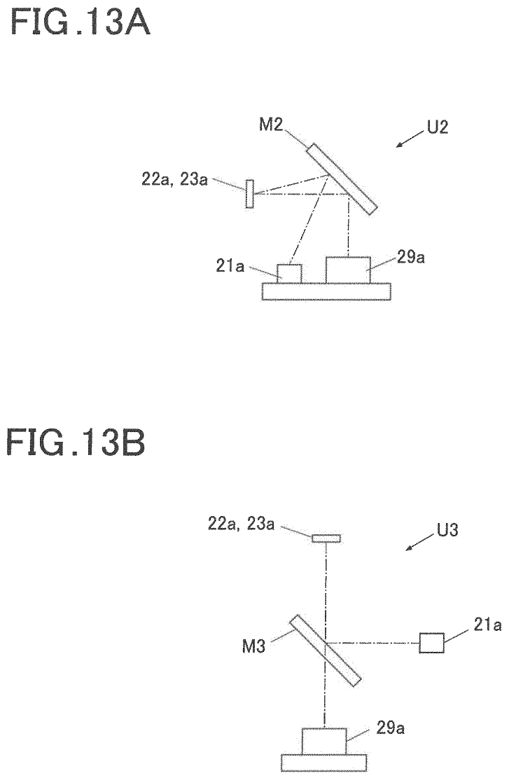

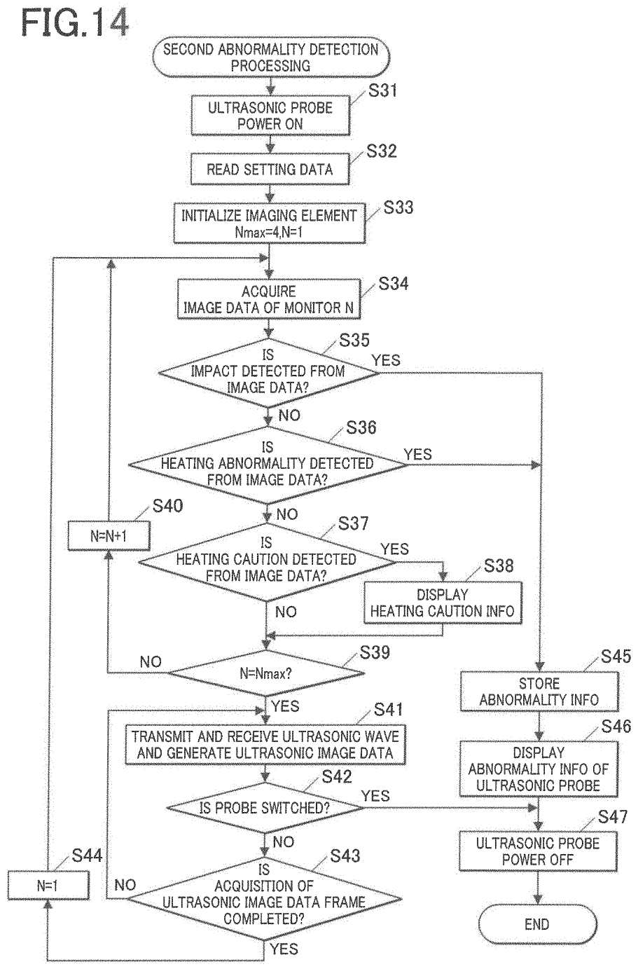

A fifth embodiment according to the present invention will be described with reference to FIG. 12 to FIG. 14. FIG. 12 is a block diagram showing a functional configuration of an ultrasonic diagnosis device 100D. FIG. 13A is a view showing a sensor unit U2. FIG. 13B is a view showing a sensor unit U3. FIG. 14 is a flowchart showing second abnormality detection processing.

In the present embodiment, an ultrasonic diagnosis device 100D shown in FIG. 12 is used in place of the ultrasonic diagnosis device 100C of the fourth embodiment. In the ultrasonic diagnosis device 100D, components similar to the components of the ultrasonic diagnosis device 100C are denoted with the same reference signs and description of the components is omitted.

The ultrasonic diagnosis device 100D includes an ultrasonic probe 2D and an ultrasonic diagnosis device main body 1D. The ultrasonic probe 2D includes a head section 2D1, a cable 3, and a connector 4D. The head section 2D1 includes a vibrator unit 2a, light sources 21a and 21b, heat detection elements 22a and 22b, impact detection elements 23a and 23b, and imaging elements 29a and 29b which constitute a state monitor.

The imaging element 29a includes a charge-coupled device (CCD), a complementary metal-oxide semiconductor (CMOS), and the like, and images a subject (the heat detection element 22a or the impact detection element 23a), generates image data including a plurality of color pixels, and outputs the data to a state judgment processor 46. The image data output from the imaging element 29a may be a video signal of a system of National Television System Committee (NTSC) or the like. It is considered that the light source 21a, the heat detection element 22a, the impact detection element 23a and the imaging element 29a constitute a set of monitors which has an identification number 1. It is also considered that the light source 21b, the heat detection element 22b, the impact detection element 23b and the imaging element 29b constitute a set of monitors which has an identification number 2.

The light source 21a and the imaging element 29a may constitute the sensor unit U1 shown in FIG. 4, in which the color sensor 24a is replaced with the imaging element 29a. Furthermore, the light source 21a and the imaging element 29a may constitute the sensor unit U2 shown in FIG. 13A or the sensor unit U3 shown in FIG. 13B. The sensor unit U2 includes the light source 21a, the imaging element 29a, and a mirror M2. The mirror M2 is disposed on an optical axis between the subject (the heat detection element 22a or the impact detection element 23a) and the imaging element 29a, and the optical axis is bent by 90 degrees. For example, light is incident on the imaging element 29a along the same axis as the optical axis from the subject. Alternatively, the sensor unit U3 includes the light source 21a, the imaging element 29a, and a mirror M3 as a half mirror. In the sensor unit U3, the light source 21a is disposed on an optical axis obtained by bending an optical axis of a subject by 90 degrees, and the light source 21a is operated as epi-illumination via the mirror M3.

The imaging element 29b has a configuration similar to the configuration of the imaging element 29a, and images the subject (the heat detection element 22b or the impact detection element 23b), generates image data, and outputs the data to the state judgment processor 46. It is considered that the light source 21b, the heat detection element 22b, the impact detection element 23b and the imaging element 29b constitute a set of monitors which has an identification number 2.

As shown in FIG. 12, the connector 4D includes light sources 41a and 41b, impact detection elements 42a and 42b, imaging elements 45a and 45b constituting a state monitor, a setting memory 44, and the state judgment processor 46.

The setting memory 44 stores setting data including a type of ultrasonic probe 2D and corresponding to identification information of the heat detection elements 22a and 22b and the impact detection elements 23a, 23b, 42a and 42b which constitute an abnormality detector. As the setting data, there is stored each setting data that is threshold information of a target area of an abnormality detection target and a color indicating an abnormality in the target area in the image data imaged and obtained to judge whether or not the image data obtained through the imaging by the imaging element 29a, 29b, 45a, or 45b indicates (occurrence of) an impact, (occurrence of) a heating abnormality, or (occurrence of) a heating caution. The heating caution indicates that any heating abnormality is not present but heat is so high that caution is required.

The imaging elements 45a and 45b have a configuration similar to the configuration of the imaging elements 29a and 29b, and each of the elements images a subject (the impact detection element 42a or 42b), generates the image data which is imaged, and outputs the data to the state judgment processor 46. It is considered that the light source 41a, the impact detection element 42a and the imaging element 45a constitute a set of monitors which has an identification number 3. It is also considered that the light source 41b, the impact detection element 42b and the imaging element 45b constitute a set of monitors which has an identification number 4.

The state judgment processor 46, in accordance with control of a controller 11D, drives the light sources 21a, 21b, 41a and 41b and the imaging elements 29a, 29b, 45a, and 45b, judges whether or not the image data input from the imaging element 29a, 29b, 45a, or 45b indicates a state of the abnormality (the impact, the heating abnormality or the heating caution), by use of the setting data read from the setting memory 44, and stores abnormality information in the setting memory 44.

The ultrasonic diagnosis device main body 1D includes the controller 11D, a transceiver 12, a switching unit 13, a display 15, and an operation unit 16. The controller 11D is similar to the controller 11 of FIG. 2, but causes the state judgment processor 46 to drive the light sources 21a, 21b, 41a and 41b and the imaging elements 29a, 29b, 45a and 45b, to acquire the image data, to judge the abnormality and to store the abnormality information in the setting memory 44. Furthermore, it is considered that a second abnormality detection program to execute the second abnormality detection processing is stored in a ROM of the controller 11D.

Next, an operation of the ultrasonic diagnosis device 100D will be described. In the ultrasonic diagnosis device 100D, for example, the controller 11D receives a trigger generated by connecting the ultrasonic probe 2D to the ultrasonic diagnosis device main body 1D and inputting an instruction for generation and display of the ultrasonic image via the operation unit 16 by an operator, and the controller executes the second abnormality detection processing in accordance with the second abnormality detection program stored in the ROM. Also in this case, the state judgment processor 46 is described as a subject in steps in which the controller 11D controls and operates the state judgment processor 46.

As shown in FIG. 14, steps S31, S32 and S33 are similar to the steps S11, S12 and S13 of FIG. 6. However, in the step S33, the state judgment processor 46 initializes the imaging elements 29a and 29b, and the controller 11D substitutes 4 for identification number Nmax of the monitor.

Then, the state judgment processor 46 drives a monitor N, and acquires the image data imaged and output from the imaging element of the monitor N (step S34). Then, the state judgment processor 46 analyzes an image of the image data acquired in the step S34 by use of the setting data for impact detection which is read in the step S32, and judges whether or not colored ink of the impact detection element in the image is diffused, to judge whether or not the impact is detected (step S35). In the step S35, as the image analysis to judge whether or not the colored ink of the impact detection element of the target area in the image is diffused, for example, it is judged whether or not a pixel of a color of the threshold information is included in the target area of the impact detection element of the setting data.

When any impact is not present (the step S35; NO), the state judgment processor 46 analyzes the image of the image data acquired in the step S14 by use of the setting data for heating abnormality detection which is read in the step S32, and judges whether or not the heat detection element in the image indicates a color of the heating abnormality, to judge whether or not the heating abnormality is detected (step S36). In the step S36, as the image analysis to judge whether or not the heat detection element in the image shows the color of the heating abnormality, for example, it is judged whether or not a pixel of a color of the threshold information is included in the target area of the impact detection element of the setting data.

When any heating abnormality is not present (the step S36; NO), the state judgment processor 46 analyzes the image of the image data acquired in the step S14 by use of the setting data for heating caution detection which is read in the step S32, and judges whether or not the heat detection element in the image shows a color of the heating caution, to judge whether or not the heating abnormality is detected (step S37). In the step S37, as the image analysis to judge whether or not the heat detection element in the image shows the color of the heating caution, for example, it is judged whether or not the pixel of the color of the threshold information is included in the target area of the impact detection element of the setting data.

Step S38 is similar to the step S18 of FIG. 6. After the step S38 is executed or when any heating abnormality is not present (the step S37; NO), the controller 11D judges whether or not the variable N=Nmax (here, Nmax=4) (step S39). Steps S40 to S44 are similar to the steps S20 to S24 of FIG. 6.

When the impact is present (the step S35; YES) or when the heating abnormality is present (the step S36;

YES), the state judgment processor 46 associates the image data acquired in the step S34 with a type of abnormality (the impact or the heating abnormality), and the current variable N (N=1: the heat detection element 22a or the impact detection element 23a, N=2: the heat detection element 22b or the impact detection element 23b, N=3: the impact detection element 42a, or N=4: the impact detection element 42b), and the state judgment processor stores the data in the setting memory 44 (step S45). Steps S46 and S47 are similar to the steps S26 and S27 of FIG. 6.

As described above, according to the present embodiment, the ultrasonic probe 2D includes the imaging elements 29a, 29b, 45a and 45b which image the heat detection elements 22a and 22b and the impact detection elements 23a, 23b, 42a and 42b and output the image data. Consequently, an internal state of the ultrasonic probe 2D can be monitored with the image data, and a change of the internal state can be securely grasped.

Furthermore, the ultrasonic probe 2D includes the state judgment processor 46. Consequently, processing burdens on the ultrasonic diagnosis device main body 1D can be decreased. Furthermore, even if the ultrasonic diagnosis device main body 1D is not connected, the ultrasonic probe 2D can perform diagnosis by sending a diagnosis control signal to the state judgment processor 46 from a testing control device such as a personal computer.

Note that the above description of each embodiment merely illustrates the preferred ultrasonic probe and ultrasonic diagnosis device according to the present invention, and the present invention is not limited to this embodiment.

At least two of the above respective embodiments may be suitably combined. For example, the color sensors of the first to fourth embodiments may be replaced with the imaging elements.

Furthermore, in each embodiment described above, the configuration example including two or four monitors has been described, but the present invention is not limited to this example as long as at least one monitor is provided.

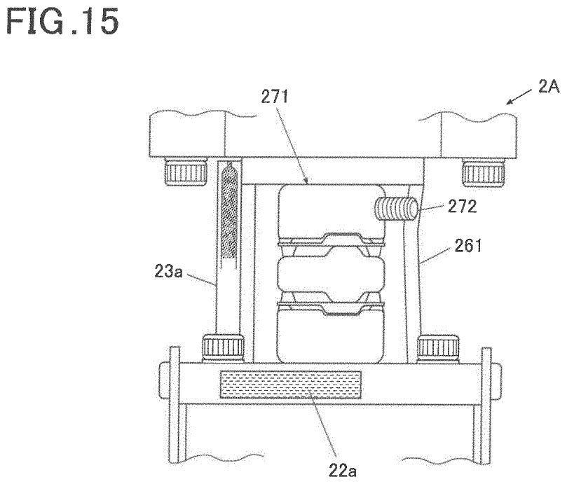

Additionally, in the second embodiment, it has been described that the color sensor outputs the wavelength-specific light amount data corresponding to the heat detection element and the impact detection element, and the state judgment processor judges the abnormality (the impact, the heating abnormality or the heating caution) from the image data obtained by the imaging, but the present invention is not limited to this embodiment. FIG. 15 is a view showing an ultrasonic probe 2A in a state where a frame 261 is deformed. In the second embodiment, it is explained that, as shown in FIG. 15, an imaging element disposed images the heat detection element and the impact detection element and outputs image data, in place of the color sensor. It is also explained that, as shown in FIG. 15, the frame 261 of the ultrasonic probe 2A is deformed in response to an impact. In the ultrasonic diagnosis device 100A, when processing similar to the second abnormality detection processing of FIG. 6 is executed and the impact is generated to deform the frame 261, in the step S45, the image data including the deformed frame 261 is stored in the setting memory 44. In the step S35, the state judgment processor 14A may analyze the image of the image data acquired in the step S34 by use of the setting data for the impact detection which is read in the step S32, and may judge whether or not the colored ink of the impact detection element in the image is diffused and whether or not the frame in the image is deformed, to judge whether or not the impact is detected.

Furthermore, in the third embodiment, it is explained that the color sensor outputs the wavelength-specific light amount data corresponding to the heat detection element and the impact detection element, the acceleration sensor outputs the acceleration data, and the state judgment processor judges the abnormality (the impact, the heating abnormality or the heating caution) from the wavelength-specific light amount data and the acceleration data, but the present invention is not limited to this configuration.

The ultrasonic probe including the drive controller and the driving unit may include the acceleration sensor. For example, it is assumed that as setting data, a threshold value of the acceleration data indicating an abnormal vibration of the driving unit is stored in the setting memory 25. In the step S15 of the first abnormality detection processing, the state judgment processor 14B may judge whether or not the wavelength-specific (R, G, or B) light amount data acquired in the step S14 is within the range of the R, G, or B setting data, by use of the setting data of the acceleration for the abnormal vibration of the driving unit which is read in the step S12, may judge whether or not the acceleration data acquired in the step S14 is in excess of the threshold value, by use of the setting data of the acceleration for the impact detection, and may judge whether or not the acceleration data for the abnormal vibration of the driving unit which is acquired in the step S14 is in excess of the threshold value, by use of the setting data of the acceleration for the abnormal vibration of the driving unit. In response to the judgments, the state judgment processor may judge whether or not the impact and the abnormal vibration are detected. According to this configuration, the impact can be detected in real time, the abnormal vibration of the driving unit can be detected, and reliability of the detection during the abnormality can further improve.

Furthermore, in each embodiment described above, for the detection of the impact and the heating abnormality, the color sensor or the imaging element monitors both the impact detection element and the heat detection element, and the state judgment processor judges the abnormality, but the present invention is not limited to this configuration. For example, in each embodiment described above, for the detection of the impact and the abnormality, the color sensor or the imaging element may monitor the impact detection element and the state judgment processor may judge the abnormality.

Additionally, in each embodiment described above, the setting data for the abnormality judgment or the like is stored in the setting memory 25 or 44 in the ultrasonic probe, but the present invention is not limited to this configuration. In the above respective embodiments, the ultrasonic diagnosis device main body may include the setting memory. It is explained that a plurality of pieces of setting data corresponding to the identification information including a plurality of ultrasonic probe types are stored in the setting memory. Furthermore, it is explained that the connector of the ultrasonic probe includes a memory in which the identification information of the ultrasonic probe is stored. The state judgment processor reads, from the memory, the identification information of the ultrasonic probe connected to the ultrasonic diagnosis device main body, reads the setting data corresponding to the identification information, and uses the data in the abnormality detection or the like.

Furthermore, detailed configurations and detailed operations of the respective components constituting the ultrasonic diagnosis devices 100, 100A, 100B, 100C and 100D in the above described embodiments can be suitably changed without departing from the gist of the present invention.

Although embodiments of the present invention have been described and illustrated in detail, the disclosed embodiments are made for purposes of illustration and example only and not limitation. The scope of the present invention should be interpreted by terms of appended claims.

The entire disclosure of Japanese Patent Application No. 2018-099251, filed on 24th of May, 2018, is incorporated herein by reference in its entirety.

What is claimed is:

1. An ultrasonic probe which transmits and receives an ultrasonic wave, comprising:
a vibrator which transmits the ultrasonic wave in response to a transmission signal, and generates a reception signal of the reflected ultrasonic wave;
at least one abnormality detector which is provided in the ultrasonic probe, and detects an abnormality and indicates the abnormality in color; and
a state monitor which is provided in the ultrasonic probe, and monitors a state of the abnormality detector by the color, and outputs monitoring information.

2. The ultrasonic probe according to claim 1, wherein the abnormality detector has an impact detection element which diffuses colored ink so that the colored ink is to be monitored, in response to an impact.

3. The ultrasonic probe according to claim 1, wherein the state monitor has a color sensor which monitors the abnormality detector and outputs wavelength-specific light amount data as the monitoring information.

4. The ultrasonic probe according to claim 1, wherein the abnormality detector has a heat detection element which changes in color in response to a surface temperature based on heat.

5. The ultrasonic probe according to claim 1, wherein the abnormality detector has an irreversible abnormality detector which keeps a detected state upon detecting the abnormality.

6. The ultrasonic probe according to claim 1, wherein the state monitor simultaneously monitors a plurality of the abnormality detectors.

7. The ultrasonic probe according to claim 6, wherein the plurality of abnormality detectors indicate the detection of the abnormality in mutually different colors.

8. The ultrasonic probe according to claim 1, wherein the state monitor has an imaging element which images the abnormality detector and outputs image data as the monitoring information.

9. The ultrasonic probe according to claim 1, comprising a driving unit which drives the vibrator and transmits the driving, wherein
the driving unit shows a different color in response to a driving state, and
the state monitor monitors the abnormality detector and the driving unit, and outputs the monitoring information.

10. The ultrasonic probe according to claim 1, comprising:
a head section having the vibrator;
a cable connected to the head section; and
a connector connected to the cable and connected to a side of a main body of an ultrasonic diagnosis device, wherein each of the head section and the connector has the abnormality detector and the state monitor.

11. The ultrasonic probe according to claim 1, comprising an acceleration detector which detects an acceleration of the ultrasonic probe.

12. The ultrasonic probe according to claim 1, comprising a first storage which stores threshold information which corresponds to identification information of the ultrasonic probe, and is for use in judgment of the abnormality from the monitoring information.

13. An ultrasonic diagnosis device comprising:
the ultrasonic probe according to claim 1;
a transceiver which inputs the transmission signal into the vibrator, and acquires the reception signal; and
a hardware processor which forms ultrasonic image data from the input reception signal, judges the abnormality of the ultrasonic probe from the output monitoring information, and stops an operation of the ultrasonic probe when the abnormality is judged.

14. An ultrasonic diagnosis device comprising:
the ultrasonic probe according to claim 12;
a transceiver which inputs the transmission signal into the vibrator, and acquires the reception signal; and
a hardware processor which forms ultrasonic image data from the input reception signal, judges the abnormality of the ultrasonic probe from the threshold information stored in the first storage and the output monitoring information, and stops an operation of the ultrasonic probe, when the abnormality is judged.

15. The ultrasonic diagnosis device according to claim 13, wherein the hardware processor stops transmission and reception of a signal from and to the ultrasonic probe, and power supply to the ultrasonic probe, when the abnormality is judged.

16. The ultrasonic diagnosis device according to claim 13, wherein the ultrasonic probe comprises a second storage, and
the hardware processor stores, in the second storage, abnormality information including monitoring information corresponding to the abnormality, when the abnormality is judged.

17. The ultrasonic diagnosis device according to claim 13, wherein the hardware processor displays, in a display, abnormality information indicating the abnormality, when the abnormality is judged.

18. The ultrasonic diagnosis device according to claim 13, wherein the hardware processor and the state monitor are connected via a network.

19. The ultrasonic diagnosis device according to claim 13, wherein the hardware processor automatically judges the abnormality of the ultrasonic probe from the output monitoring information, when the ultrasonic probe is connected to the hardware processor.

20. The ultrasonic diagnosis device according to claim 13, wherein the ultrasonic probe comprises the hardware processor which performs the judgment.
